(19) United States
(12) Patent Application Publication
(10) Pub. No.: US 2025/0003993 A1
(43) Pub. Date: Jan. 2, 2025

(54) METHODS AND KITS FOR IDENTIFYING AND QUANTIFYING STORAGE-INDUCED MICROERYTHROCYTES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); UNIVERSITÉ PARIS CITÉ, Paris (FR); UNIVERSITÉ DES ANTILLES ET DE LA GUYANE, Pointe-à-Pitre (FR); FONDATION IMAGINE, Paris (FR)

(72) Inventors: Pascal AMIREAULT, Paris (FR); Sandy PELTIER, Paris Cedex 15 (FR); Mickaël MARIN, Paris Cedex 15 (FR); Pierre BUFFET, Paris Cedex 15 (FR); Michaël DUSSIOT, Paris (FR); Camille ROUSSEL, Paris Cedex 15 (FR)

(21) Appl. No.: 18/709,687

(22) PCT Filed: Dec. 13, 2022
(86) PCT No.: PCT/EP2022/085632

§ 371 Date: May 13, 2024

(30) Foreign Application Priority Data
Dec. 14, 2021 (EP) 21306765.5

(51) Int. Cl. G01N 33/80; G01N 15/01; G01N 15/10; G01N 15/14

(52) U.S. Cl. CPC G01N 33/80; G01N 15/1459; G01N 2015/012; G01N 2015/1006

(57) ABSTRACT

Refrigerated storage of red cell concentrates (RCC) for transfusion is associated with the accumulation of various alterations to the red blood cells (RBCs). Among these, a subpopulation of small RBCs defined as storage-induced microerythrocytes (SMEs) accumulates during storage. The SMEs subpopulation correlates with transfusion recovery. Quantification of this morphologically-altered RBC subpopulation using flow cytometry would be a valuable tool to evaluate RCC quality. In the present invention, RBC obtained at the beginning or at the end of storage from RCC stored in SAGM in blood bank conditions were treated with a carboxyfluorescein diacetate succinimidyl ester (CFDA-SE) staining protocol and were finally analysed by flow cytometry to assess the intensity of CFSE staining. The inventors observed the accumulation of a CFDA-SE^{high} subpopulation by flow cytometry that accounted for 0.8% and 36.3% at day 3 and 42 of storage, respectively. Images confirmed that the CFDA-SE^{high} subpopulation mostly contains SMEs. Thus SMEs can now be simply quantified using a common fluorescent dye and a standard flow cytometer.

### FIELD OF THE INVENTION

The present invention is in the field of medicine, in particular haematology.

### BACKGROUND OF THE INVENTION

During hypothermic pre-transfusion storage, part of the RBC metabolism shifts from glycolysis to the pentose phosphate pathway after 10-14 days of storage, leading to a progressive decrease of the intracellular ATP level (D'Alessandro et al, Transfusion, 2015; Bordbar A et al, Transfusion, 2016). The fragilized RBC is thus less capable of coping with the oxidative stress present in the bag further contributing to fragilize the RBC integrity (Reisz J A et al, Blood, 2016). These metabolic and oxidative stresses contribute to the progressive modifications of RBC properties (Yoshida T et al, Blood Transfus, 2019). Among those, RBC morphology is a well-documented and key RBC property altered during storage.

A number of techniques are used to explore RBC morphology. The pioneer work of Bessis and his collaborators using scanning electron microscopy defined seven RBC morphology classes (Bessis M, Nouvel Rev Fran Hematol, 1972) (Bessis M, livre Corpuscules, 1974). This technique is still used to assess RBC morphology during storage (Berezina, 2012; D'Alessandro, haematologica, 2012; Blasi, 2012; Antonelou M H, Journal of Proteomics, 2012; Roussel, Morel, 2021; Zehnder L et al, Vox Sang, 2008).

Digital holographic microscopy was recently used to explore RBC morphology along storage (Bardyn, Blood Transfus, 2017). Light microscopy can also be used to observe fixed (Högman, Vox Sang, 1985; Tchir, Transfusion, 2013; Reinhart, Transfusion, 2015) or unfixed cells in a physiological medium (Lu and shevkoplyas, Transfusion, 2020; Roussel, Transfusion, 2017). RBC morphology is indeed sensitive to RBC intrinsic properties and to the suspension medium (Bessis M, Nouvel Rev Fran Hematol, 1972). Imaging flow cytometry rapidly acquires tens of thousands of images of unfixed cells in a physiological medium and identifies a well-demarcated subpopulation of morphologically altered and smaller RBCs, comprising type III echinocytes, spheroechinocytes, and spherocytes (Roussel, 2017). These small RBCs, defined as storage-induced micro-erythrocytes (SMEs), accumulate during storage, reaching a mean proportion of 25% of the entire RBC population at day 42 of storage and negatively correlate with post-transfusion recovery in healthy volunteers (Roussel, 2021). Furthermore, the Imaging Flow Cytometry capacity to discriminate fluorescently-stained cells allowed to observe their clearance from circulation after ex vivo perfusion in a human spleen and in vivo transfusion in mouse model (Roussel, 2021). Quantification of morphologically-altered RBCs is thus a relevant storage quality marker that identifies the RBC subpopulation targeted for rapid post-transfusion clearance. Quantification of morphologically-altered RBCs using flow cytometry would be simpler. Also, obtaining purified preparations of these cells could allow to investigate the intrinsic properties of RBC that lead to their clearance.

### SUMMARY OF THE INVENTION

The present invention is defined by the claims. In particular, the present invention relates to methods and kits for identifying and quantifying storage-induced microerythrocytes (SME).

### DETAILED DESCRIPTION OF THE INVENTION

Refrigerated storage of red cell concentrates (RCC) for transfusion is associated with the accumulation of various alterations to the red blood cells (RBCs). Among these, a subpopulation of small RBCs, comprising type III echinocytes, sphero-echinocytes, and spherocytes and defined as storage-induced micro-erythrocytes (SMEs) accumulate to reach approximately 25% of RBC on day 42 of storage. The SMEs subpopulation is variable between donors, is cleared rapidly after transfusion, correlates with transfusion recovery and can be rapidly and objectively quantified using imaging flow cytometry. Selection and quantification of this morphologically-altered RBC subpopulation using flow cytometry would be a valuable tool to evaluate RCC quality but a selective stain is lacking. The inventors obtained RBC at the beginning or at the end of storage from RCC stored in SAGM in blood bank conditions. The RBC were then treated with a carboxyfluorescein diacetate succinimidyl ester (CFDA-SE) staining protocol and were finally analysed by flow cytometry to assess the intensity of CFDA-SE staining. In parallel, SMEs were quantified by imaging flow cytometry. The inventors observed the accumulation of a CFDA-SE^{high} subpopulation by flow cytometry that accounted for 0.8% and 36.3% at day 3 and 42 of storage, respectively. Imaging flow cytometry showed a mean projected area of 45.6 μm² and 78.2 μm² for CFDA-SE^{high} and CFDA-SE^{low} subpopulations, respectively. Images confirmed that the CFDA-SE^{high} subpopulation mostly contains SMEs while the CFDA-SE^{low} subpopulation mostly contains type I and II echinocytes and discocytes. Similar numbers of SMEs were quantified by imaging flow cytometry (based on projected surface area of RBC) and by conventional flow cytometry (based on CFDA-SE intensity). CFDA-SE^{high} and CFDA-SE^{low} subpopulations could be sorted by flow cytometry and contained >95% and <5% SMEs, respectively, by imaging flow cytometry and scanning electron microscopy. SMEs can now be simply quantified using a common fluorescent dye and a standard flow cytometer. This simple staining protocol enables the specific sorting of SMEs, a useful tool to further characterize this RBC subpopulation targeted for clearance after transfusion.

The present invention relates to a method for detecting the presence of storage-induced microerythrocytes (SMEs) in a red blood cell containing composition comprising the steps of i) staining the composition with an amount of a cell permeable dye, ii) incubating the stained composition for a sufficient period of time and under conditions suitable for allowing a bimodality of staining to appear in the red blood cells, and iii) distinguishing the cells by their intensity of staining wherein the subpopulation of SMEs are characterized by a high intensity of staining.

As used herein, the term “erythrocytes” or “red blood cells” or “RBCs” has its general meaning in the art and refers to highly-specialized cells responsible for delivery of oxygen to, and removal of carbon dioxide from, metabolically-active cells via the capillary network. They are shaped as biconcave discs and average about 8-10 microns in diameter.

A used herein, the term “storage-induced microerythrocytes” or “SMEs” has its general meaning in the art and refers to a subpopulation of small RBCs, comprising type III echinocytes, spheroechinocytes, and spherocytes. During storage, RBCs indeed show various stages of morphological deterioration, including echinocyte 1 (E1), echinocyte 2 (E2), echinocyte 3 (E3), sphero-echinocyte (SE), spherocyte(S) and stomatocyte (ST).

As used herein, the term “red blood cell containing composition” means whole blood, red blood cell concentrates and any other composition that contains red blood cells. Typically, the red blood cell containing composition is an individual stored red blood cell (RBC) unit. Other than red blood cells, the composition can also contain a biologically compatible solution, such as SAGM, PAGGSM, ARC-8, Nutricell (AS-3), ADSOL (AS-1), Optisol (AS-5) or RAS-2 (Erythrosol), and one or more cellular blood components, one or more blood proteins, or a mixture of one or more cellular blood components and/or one or more blood proteins. Such compositions may also contain a liquid blood component, such as plasma. Typically, the red blood cell containing composition was stored for a period of 1 to 42 days at a temperature of 2 to 6° C., preferably at 4° C.

As used herein, the term “cell permeable dye” has its general meaning in the art and refers to a dye that freely diffuses through the membranes of live cells. In some embodiments, the dye is a fluorescent dye. As used herein, the term “fluorescent dye” means a dye which absorbs light at a first wavelength and emits at second wavelength which is longer than the first wavelength. Fluorescent dyes typically include fluorescent compounds having a chemically reactive group that facilitates attachment of dye to a conjugate molecule. Numerous fluorescence dyes useful for selective labeling of viable cells are described in the art. In particular, the dye is an “amine tracer” wherein upon reaction with amine-containing residues of intracellular proteins, the resulting dye-protein adducts are well retained in cells.

In some embodiments, the dye is a succinimidyl ester-based dye that binds to intracellular proteins. In some embodiments, the dye is a succinimidyl ester-based dye that binds to the amine groups of cellular proteins. In some embodiments, the dye is a carboxyfluorescein succinimidyl ester dye that binds to amine groups of cellular proteins. For instance, succinimidyl esters can be prepared simply by reacting fluorescent dyes bearing carboxyl groups with N-hydroxysulfosuccinimides. In some embodiments, the dye is selected from the group consisting of carboxyfluorescein diacetate succinimidyl ester (CFDA-SE), carboxyfluorescein succinimidyl ester (CFSE), carboxyeosin diacetate succinimidyl ester or any derivatives thereof. In some embodiments, the dye is a carboxyfluorescein succinimidyl ester dye. For instance, the dye can also be selected from CellTrace™ dyes (e.g. CellTrace Violet™, CellTrace™ Far Red). In some embodiments, the dye is CellTrace Violet (CTV) dye. In some embodiments, the dye is CellTrace Far Red dye. In some embodiments, the dye is carboxyfluorescein diacetate succinimidyl ester (CFDA-SE, or, alternatively, CFSE) that is excitable with 488 nm laser light to give a bright green fluorescence with a maximum emission at 517 nm. In some embodiments, the dye is carboxyfluorescein diacetate succinimidyl ester (CFDA-SE, or, alternatively, CFSE) that is excitable with 492 nm laser light to give a bright green fluorescence with a maximum emission at 517 nm. In some embodiments, the dye is CellTrace Violet (CTV) dye that is excitable with 405 nm laser light to give a bright fluorescence with a maximum emission at 550 nm. The term “CFDA-SE” has its general meaning in the art and refers to 2,5-Dioxopyrrolidin-1-yl 3′,6′-bis(acetyloxy)-3-oxo-3H-spiro[[2]benzofuran-1,9′-xanthene]-6-carboxylate.

In general, the dye is provided to the cells at a concentration ranging from about 0.01 μM to about 1 μM, preferably 0.05 μM. Typically, the incubation step is carried out at 37° C. and for at least 10, 15, 20, 25 or 30 minutes preferably 20 minutes. Typically, the cells once stained are centrifugated and washed and then incubated in an appropriate culture medium (e.g. RPMIc) for at least 4 h, typically overnight. Following incubation, the cells are centrifuged, resuspended in a fresh culture medium and stored until analysis.

After being contacted with the fluorescent dye, the composition is excited by a light source capable of producing light at or near the wavelength of maximum absorption of the fluorescent complex, such a laser, an arc lamp, an ultraviolet or visible wavelength emission lamp. Any apparatus or device that can measure the total fluorescence of a sample can be used in this invention including flow cytometers. Typically, the fluorescence emitted by the fluorescent dye is measured by means of a flow cytometer, such as a Fluorescence Activated Cell Sorter (FACS) machine. As used herein, the term “fluorescence activated cell sorting” or “FACS” refers to a method by which the individual cells of a sample are analysed and sorted according to their optical properties (e.g., light absorbance, light scattering and fluorescence properties, etc.) as they pass in a narrow stream in single file through a laser beam. Fluorescence-activated cell sorting is a specialized type of flow cytometry. It provides a method for sorting a heterogeneous mixture of biological cells into two or more containers, one cell at a time, based upon the specific light scattering and fluorescent characteristics of each cell. It is a useful scientific instrument as it provides fast, objective and quantitative recording of fluorescent signals from individual cells as well as physical separation of cells of particular interest. In a typical FACS system, the cell suspension is entrained in the centre of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell being in a droplet. Just before the stream breaks into droplets the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge. In some systems the charge is applied directly to the stream and the droplet breaking off retains charge of the same sign as the stream. The stream is then returned to neutral after the droplet breaks off. The fluorescent labels for FACS technique depend on the lamp or laser used to excite the fluorescent dye and on the detectors available.

In some embodiments, the fluorescent dye intensity is expressed as an absolute value (e.g. fluorescence intensity) or as a rate (e.g. fluorescence intensity by a section of time). In some embodiments, the fluorescence intensity is expressed as MFI. The term “MFI”, as used herein, refers to the mean or median fluorescence intensity of a population of fluorescent cells. As used herein, the expression “high intensity of staining” indicates that intensity of the staining is high by comparison with the staining intensity in whole population of cells. The term “high” thus indicates that the intensity is higher than the mean or median of intensity measured in the whole population of cells. Typically, when the CFDA-SE dye is used the population of SMEs is defined as the CFDA-SE^{high} fraction.

In some embodiments, the method of the present invention comprises the step of assessing the size of the cells. According to the present invention the SMEs are indeed characterized by a small size and a high intensity of staining. The term “small size” typically indicates that the size of the cells is lower than the mean or median of size measured in the whole population of cells.

In some embodiments, FACS brings the advantage that by using a gating strategy it is possible in the same sample to determine the fluorescent dye intensity and the size of the cells. Typically, a strategy of gating is described in the EXAMPLE for identifying the population of SMEs.

In some embodiments, the method of the present invention comprises the step of quantifying the amount of SMEs present in the red blood cell containing composition.

The method of the present invention is particularly suitable for quantifying the quality degradation of red blood cell containing compositions, and processing this data to facilitate better-informed medical decisions regarding units' respective allocation, patient suitability, and use. Typically deploying the method across a hospital's inventory or even throughout the supply chain will improve distribution, planning, and inventory control decisions. In particular, the method of the present invention is of particular interest in the context of chronically transfused patients for which the poor transfusion recovery could have a major impact. For example, transfusion-related iron overload is a major cause of morbidity and mortality in these patients, and the need to provide more transfusions because some are of lower quality or recovery could have significant adverse consequences. Thus, the method of the present invention is of a particular interest for patients suffering from haematological conditions such as β-hemoglobinopathies such as sickle cell disease or thalassemia (e.g. β-thalassemia).

Accordingly, a further object of the present invention relates to a method of assessing the storage quality of a red blood cell containing solution comprising quantifying the amount of SMEs present in the composition by the quantifying method herein disclosed wherein the amount of SMEs correlates with the quality storage.

As used herein, “storage quality” is defined as the extent of post-transfusion recovery of the stored RBCs; higher recovery is defined as higher quality. Examples of post-transfusion recovery include greater than zero and almost 100% recovery, i.e., recovery of 10%, 20%, 30%, 40%, 50%, 60%, 70%, 80%, 90%, 100%, and all percentages in between. In some embodiments, an acceptable storage quality is an average of 75% post-transfusion recovery at 24 hours, as under FDA guidelines.

Typically, the higher is the amount of SMEs present in the composition, the worse is the storage quality of the red blood cell composition. In particular, red blood cell containing composition is considered as being of good quality when at least about 70%, preferably at least about 75%, still preferably at least about 80%, 85%, advantageously 90%, 91%, 92%, 93%, 94%, and ideally 95%, 96%, 97%, 98%, 99% or 100% of the RBCs are not morphologically altered in SMEs.

In particular, the method of the present invention is also particularly suitable for predicting the transfusion recovery of a patient. It is established that when the subpopulation of SMEs was abundant in RBC concentrates, transfusion recovery is diminished. Typically, the patient is a chronically transfused patient and more particularly a patient suffering from a β-hemoglobinopathy.

Accordingly, a further object of the present invention relates to a method of predicting the transfusion recovery of a patient transfused with a red blood cell containing composition comprising quantifying the amount of SMEs present in the composition by the quantifying method herein disclosed wherein the amount of SMEs correlates with the recovery yield. More particularly, the method comprises the steps of i) quantifying the amount of SMEs present in the red blood cell containing solution, ii) comparing the amount quantified at step i) with a predetermined reference value, iii) and concluding that the patient will a have a good recovery when the amount quantified at step i) is lower than the predetermined reference value or concluding that the patient will have a poor recovery when the amount quantified at step i) is higher than the predetermined reference value.

Typically, the predetermined reference value is a threshold value or a cut-off value. A “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective quantification of SMEs in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the amount of SMEs in a group of reference, one can use algorithmic analysis for the statistic treatment of the measured amounts of SMEs in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc. In some embodiments, a cut-off value consists of a range of quantification values, e.g. centered on the quantification value for which the highest statistical significance value is found. For example, on a hypothetical scale of 1 to 10, if the ideal cut-off value (the value with the highest statistical significance) is 5, a suitable (exemplary) range may be from 4-6. For example, a patient may be assessed by comparing values obtained by measuring the amount of SMEs, where values greater than 5 reveal that the patient is at risk of having a poor recovery and values less than 5 reveal that the patient will have a good recovery. In some embodiments, a patient may be assessed by comparing values obtained by measuring the amount of SMEs and comparing the values on a scale, where values above the range of 4-6 indicate that the patient is at risk of having a poor recovery and values below the range of 4-6 indicate that the patient will have a good recovery, with values falling within the range of 4-6 indicate that further explorations are needed to conclude whether the patient is at risk of having a poor recovery.

The method of the present invention is also particularly suitable for preparing a red blood cell containing composition suitable for transfusion comprising i) detecting the presence of SMEs in red blood cell containing composition and ii) separating the SMEs from blood cell containing composition. Any sorting method as described above (e.g. FACS) can be used for said preparation.

Also encompassed within the scope of the invention are test kits for performing the methods of the present invention. Complete test kits contain solutions and devices quantifying the amount of SMEs present in a red blood cell containing composition. For example, the test kit contains a 96, 384, or 1536 well plate for high throughput sample purification, and/or solutions for staining the population of red blood cells with the cell permeable dye. Generally, the test kits of the invention contain one or more of the following: (1) one or more containers (2) one or more solution containing the cell permeable dye for staining the red blood cells; (3) instructions for practicing the methods described herein; (4) one or more assay component; and (5) packaging materials. In some embodiments, the kits include in addition to one or more of the, buffers, reagents, chemical agents, functionalization reagents, enzymes, detection agents, control materials, or the like, among others. Other items which may be provided as part of the test kit include solid surface syringes, pipettes, cuvettes, and containers.

The invention will be further illustrated by the following figures and examples. However, these examples and figures should not be interpreted in any way as limiting the scope of the present invention.

### FIGURES

FIG. 1: Bimodality in Carboxyfluorescein diacetate succinimidyl ester (CFDA-SE) reactivity in RBC stored in blood bank conditions.

Representative frequency plots of fluorescence intensity for RBC stored for 42 days in SAGM, either immediately after CFDA-SE staining (grey histogram) and during the 24 hours of incubation at 37° C. (black line).

FIG. 2: CFDA-SE^{high} cells correspond to morphologically-altered RBCs that accumulate during pretransfusion storage.

(A) Representative CFDA-SE intensity frequency plots of unstained (grey histogram) and CFDA-SE stained (black line) long-stored RBCs, from imaging flow cytometry analysis, allowing to select CFDA-SE^{low} (dotted line) and CFDA-SE^{high} (dashdotted line) subpopulations by the nadir point of the bimodality of frequency plots. (B) Comparison of the mean CFDA-SE fluorescence intensities of each subpopulation obtained from 8 RCCs stored 42 days and determined by imaging flow cytometry. (C) Representative projected surface area frequency plots of the CFDA-SE^{low} and CFDA-SE^{high} subpopulations previously selected in A. (D) Comparison of the mean projected surface area from the CFDA-SE subpopulations previously selected in B from the 8 long-stored RCCs. Representative ImageStream brightfield and fluorescence images from CFDA-SE^{high} (E) showing mostly echinocytes III, spheroechinocytes and spherocytes, and CFDA-SE^{low} subpopulations (F) showing discocytes, echinocytes I and echinocytes II. Scale bars represent 7 μm. Results are presented as mean±SD in B and D and tests of Wilcoxon for non-parametric and paired data were applied to compare groups between each other (**: p=0.0078).

FIG. 3: CFDA-SE^{high} staining allows quantification by flow cytometry of morphologically-altered RBCs along storage

(A) Representative density plots allowing quantification of CFDA-SE^{low} and CFDA-SE^{high} subpopulations along storage by flow cytometry. (B) Representative projected surface area frequency plots for unstained RBCs allowing quantification of morphologically-normal (grey line) and morphologically-altered (SME, black line) RBCs along storage by imaging flow cytometry. (C) Evolution of the proportion of the CFDA-SE^{high} (grey line) and unstained SMEs subpopulations (black line) during RCC storage (n=8). (D) Correlation between the two quantification techniques of morphologically-altered RBCs that accumulate along storage (Spearman r=0.93, p<0.0001; r²=0.88). Results in C are represented as mean±SD (vertical bars) and a two-way ANOVA, with the Geisser-Greenhouse correction followed by a Sidak's multiple comparison test, compared both techniques at each time point (*: p<0.05) or the accumulation of CFDA-SE^{high} subpopulation along storage vs day 3 (‡‡: p=0.0011; ‡‡‡‡: p<0.0001).

FIG. 4: Enriched preparations of morphologically-normal and SMEs subpopulations of stored RBCs can be obtained by flow cytometry sorting.

(A) Flow cytometry sorting gating strategy, using fluorescence intensity and morphological parameters to select CFDA-SE^{low} (grey dotted line) and CFDA-SE^{high} (black dashdotted line) RBC subpopulations. Morphological criteria comprised the forward scatter (FSC) or back scatter (BSC) signals and their respective parameter of area (A), height (H) and width (W). (B) Representative imaging flow cytometry density plots of unsorted (middle panel), sorted CFDA-SE^{low} (left panel) and CFDA-SE^{high} (right panel) subpopulations illustrating each preparation purity. (C) Scanning electron microscopy images showing the morphology of RBC content in each unsorted or sorted fractions. Scale bars represent 2 μm.

FIG. 5: Morphologically-altered RBCs that accumulate during pretransfusion storage show higher CFDA-SE staining intensity

(A) Representative projected surface area frequency plot, obtained by imaging flow cytometry, of unstained (grey histogram) and CFDA-SE stained (black line) long-stored RBCs segregating SME (dashdotted line) and normal cells (dotted line) subpopulations by the nadir point of the bimodal distribution. (B) Comparison of the mean projected surface area of SME and normal cells subpopulation determined as in A, from 8 RCCs stored 42 days. (C) Representative CFDA-SE intensity frequency plots of unstained (grey histogram) and the SME (dashdotted line) and normal cells (dotted line) subpopulations previously selected in A. (D) Comparison of the mean CFDA-SE fluorescence intensities of each SME and normal cells subpopulations determined as in C obtained from 8 RCCs stored 42 days. Results are presented as mean±SD in B and D and tests of Wilcoxon for non-parametric and paired data were applied to compare groups between each other (**: p=0.0078).

FIG. 6: Selection of the gating strategy to quantify morphologically-altered CFDA-SE^{high} and morphologically-normal CFDA-SE^{low} subpopulations of stored RBCs

(A) Representative imaging flow cytometry dot plot of projected surface area and fluorescence intensity of CFDA-stained RBCs after 42 days of storage. Selection using only fluorescence intensity (vertical dotted black bold line) shows that (B) CFDA-SE^{low} subpopulation contains some morphologically-altered RBCs and that (C) CFDA-SE^{high} subpopulation contains morphologically-normal RBCs. (D) Selection on morphological (projected surface area with imaging flow cytometry) and fluorescence (CFDA-SE intensity) criteria (oblique dotted black bold line) improves the gating specificity.

FIG. 7: A Cell Trace Violet staining protocol identifies the same subpopulation of stored RBCs as the CFDA-SE staining protocol, allowing quantification of SMEs by flow cytometry in a different fluorescence channel. (A) Representative images of CFDA-SE and Cell Trace Violet (CTV) co-stained RBCs obtained by ImageStream, BF=Brightfield. (B) Accumulation of CFDA-SE^{high} RBCs and CTV^{high} RBCs along storage (n=8). There was no significant difference. (C) Intracellular ATP content normalized by hemoglobin content in sorted subpopulations identified by CFDA-SE or CTV staining (n=3). In B and C, results are represented as mean±SEM and a two-way ANOVA followed by a Sidak's multiple comparison was performed to compare both staining at each time point or for each subpopulation, respectively. No statistically significant difference was observed.

FIG. 8: Most CFDA-SE^{high} red blood cells are rapidly cleared upon ex vivo perfusion of human spleen. Kinetics of short-stored RBCs, long-stored RBCs and their CFDA-SE^{low} and CFDA-SE^{high} subpopulations persistence in circulation during perfusion in human spleen ex vivo. Results are represented as the mean±SEM of 4 RCCs. SS=short-stored RBCs, LS=long-stored RBCs.

FIG. 9: Storage lesions are concentrated in the CFDA-SE^{high} subpopulation of long-stored RBCs. Retention rate by microsphiltration (A), dynamic adhesion experiments on endothelial cells (B), PS exposition by lactadherin staining (C), osmotic fragility by measuring NaCl concentration inducing 50% hemolysis (D) and intracellular ATP level normalized by hemoglobin content (E) were evaluated on long-stored CFDA-SE-stained RBCs sorted by flow cytometry to obtain highly enriched preparations of CFDA-SE^{low} and CFDA-SE^{high} RBCs. Unstained and unsorted CFDA-SE-stained RBCs were used as control. In C, a CTV staining protocol was used instead of the CFDA-SE staining protocol. All data are represented as individual point (mean is represented by a line). One-way ANOVA of Friedman followed by Dunn's multiple comparison test were performed for all experiments.

### EXAMPLE 1

### Methods

### RBC Concentrate Collection and Storage

Eight leukoreduced RBC concentrates provided by the Establishment Françee ais du Sang Haut de France-Normandie (French blood banking system) from healthy donors were stored in saline-adenine-glucose-mannitol (SAGM) solution at 2 to 6° C. for 42 days. Samples were aseptically collected and analyzed at defined time-points (days 3, 21, 28, 35 and 42).

### CFDA-SE Staining

RBC were washed once in phosphate buffer saline (PBS) and stained with CFDA-SE (5.5 millions RBC/mL, 0.05 μM CFDA-SE in PBS) for 20 minutes at 37° C. RBCs were then centrifuged, washed once in RPMIc (RPMI 1640 supplemented with 10% FBS, 1% Antibiotic/Antimycotic solution) and incubated overnight in RPMIc at 22 millions RBC/mL. Following incubation, RBCs were centrifuged, resuspended in a fresh RPMIc solution and stored at 4° C. until analysis.

### Flow Cytometry Analysis

CFDA-SE-stained RBCs were analyzed using FACScanto II (BD Biosciences) by recording 50,000 events. First, single cells were selected using morphological parameters (FSC-H vs FSC-A). CFDA-SE^{low} and CFDA-SE^{high} subpopulations were gated according to their size (FSC-W) and their CFDA-SE intensity (collected in FITC channel).

### Imaging Flow Cytometry Analysis

Imaging flow cytometry was performed by ImageStream X Mark II (Amnis of EMD Millipore) to determine RBC dimensions and morphology as described (ROUSSEL C. et al, Transfusion 2017). RBC were suspended at 1% hematocrit (Hct) just before acquisition in a Krebs-albumin solution (Krebs-Henseleit buffer, Sigma-Aldrich) modified with 2 g of glucose, 2.1 g of sodium bicarbonate, 0.175 g of calcium chloride dehydrate, and 5 g of lipid-rich bovine serum albumin (Albu-MAX II, Thermo Fisher Scientific) for 1 L of sterile water (pH 7.4). Images (×60 magnification) were recorded (INSPIRE software, AMNIS) by the brightfield and FITC channels to be then processed by a dedicated computer software (IDEAS [version 6.2]; Amnis). Focused cells and single cells were respectively selected using the features gradient RMS_M01_Ch01 and Aspect ratio_M01_Ch01 versus Area_M01_Ch01. Front views were selected using the feature Circularity_Object (M01, Ch01, Tight) and projected surface area was determined using the feature Area_Object (M01, Ch01, Tight). At least 6000 front views of focused single RBC/condition were analyzed. SME proportion and CFDA-SE intensity were determined independently for each donor, using the nadir of the bimodal frequency histograms as the gating boundary.

### Cell Sorting

Sorting of CFDA-SE^{low} and CFDA-SE^{high} cells was performed using MA900 Cell Sorter (Sony) with a 100 μm sorting-chip at the maximum speed of 10,000 events per second in semi-purity mode. Unsorted RBCs were selected using BSC-A vs FSC-A then CFDA-SE^{low} and CFDA-SE^{high} subpopulations were gated according to their size (FSC-W) and their CFDA-SE intensity (collected in FITC channel). Cell doublets were excluded by using FSC-H vs FSC-A and target cells were collected in tubes containing 1 mL of RPMIc, then centrifuged and resuspended in RPMIc to be stored at 4° C. until analysis.

### Scanning Electron Microscopy

Samples were fixed by incubation of minimum 24 h in a mix of 4% paraformaldehyde and 1% glutaraldehyde diluted in 0.1 M of phosphate buffer (pH, 7.3) at 4° C., then washed in phosphate buffer, and postfixed by 1-hour incubation with 2% osmium tetroxide. Samples were then fully dehydrated in a graded series of ethanol solutions and dried by hexamethyldisilazane. Finally, samples were coated with 4 nm of carbon using a GATAN PECS 682 apparatus before observation under a Zeiss Ultra plus field emission-scanning electron microscope (ZEISS).

### Statistical Analysis

Data were analyzed using GraphPad Prism version 9.2.0 for Windows (GraphPad Software, San Diego, California USA). To compare two groups of subpopulations (CFDA-SE^{low} vs CFDA-SE^{high} intensities or SME vs normal cells), Wilcoxon tests, for non-parametric and paired data, were applied. To compare means of CFDA-SE^{high} RBCs over time, two-way ANOVA with the Geisser-Greenhouse correction was performed with Sidak's multiple comparison test. Simple linear regression and a correlation test of Spearman were used to assess correlation between the two quantification techniques of CFDA-SE^{high} RBCs and SMEs. A P-value <0.05 was considered statistically significant.

### Results

### Bimodality in Carboxyfluorescein Diacetate Succinimidyl Ester (CFDA-SE) Reactivity in RBC Stored in Blood Bank Conditions

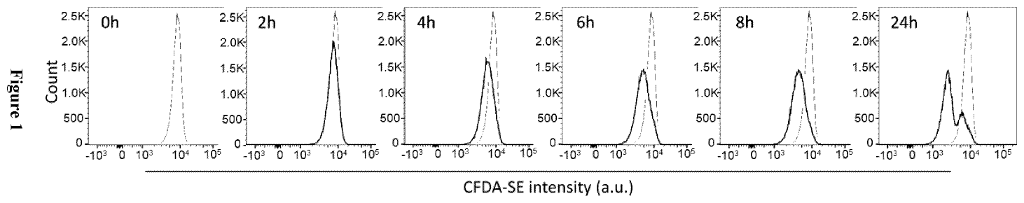

RBC, sampled from long-stored RBC concentrates, were stained using CFDA-SE and then incubated at 37° C. in RPMIc medium. Immediately after CFDA-SE staining fluorescence intensity exhibited a unimodal distribution (Oh, grey histogram, FIG. 1). Incubation of the labeled RBC at 37° C. lead to a progressive decline of fluorescence intensity in a subpopulation of RBC (black curves, FIG. 1). Following an overnight incubation, the CFDA-SE intensity showed a clear bimodality separating two RBC subpopulations (FIG. 1). The bimodality in CFSE-DA staining was not visible when fresh RBCs were observed or when RBCs were incubated at 4° C. after staining (FIG. 5A, B, C, D). These results show that the CFDA-SE staining protocol with an overnight incubation identifies a discrete RBC subpopulation present in long-stored RBC.

### CFDA-SE^{high} Cells Correspond to Morphologically-Altered RBCs that Accumulate During Pretransfusion Storage

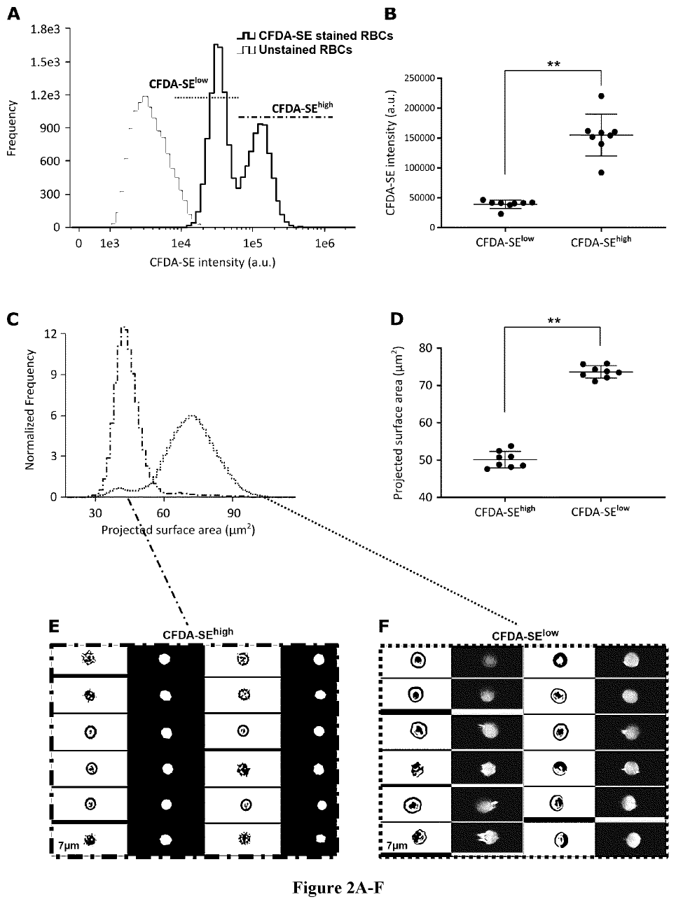

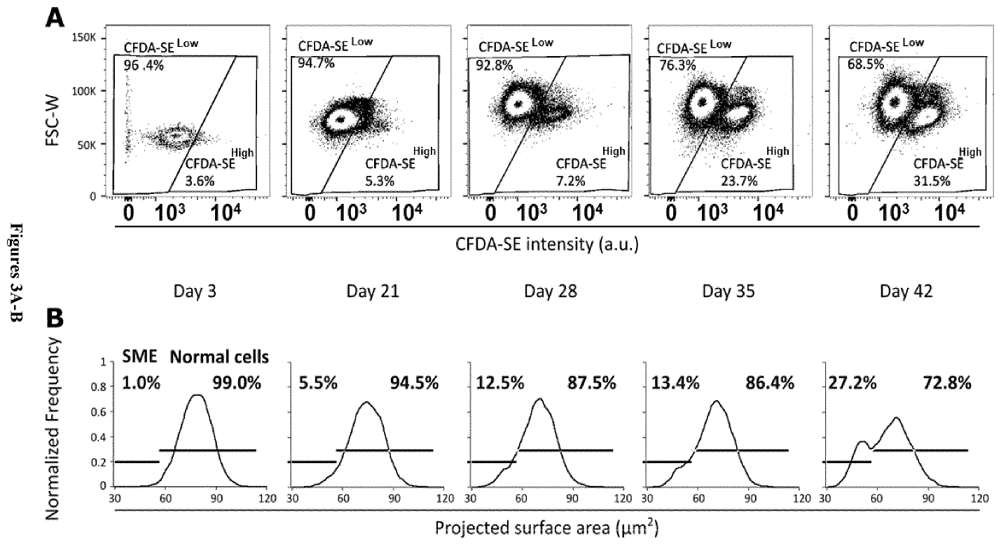

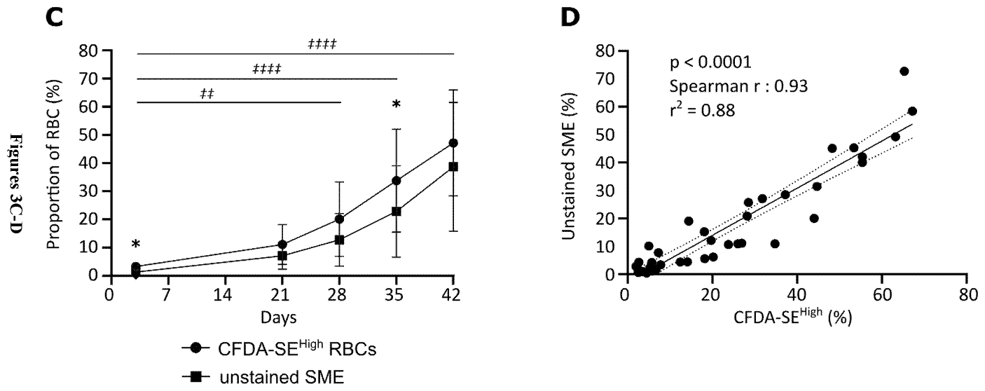

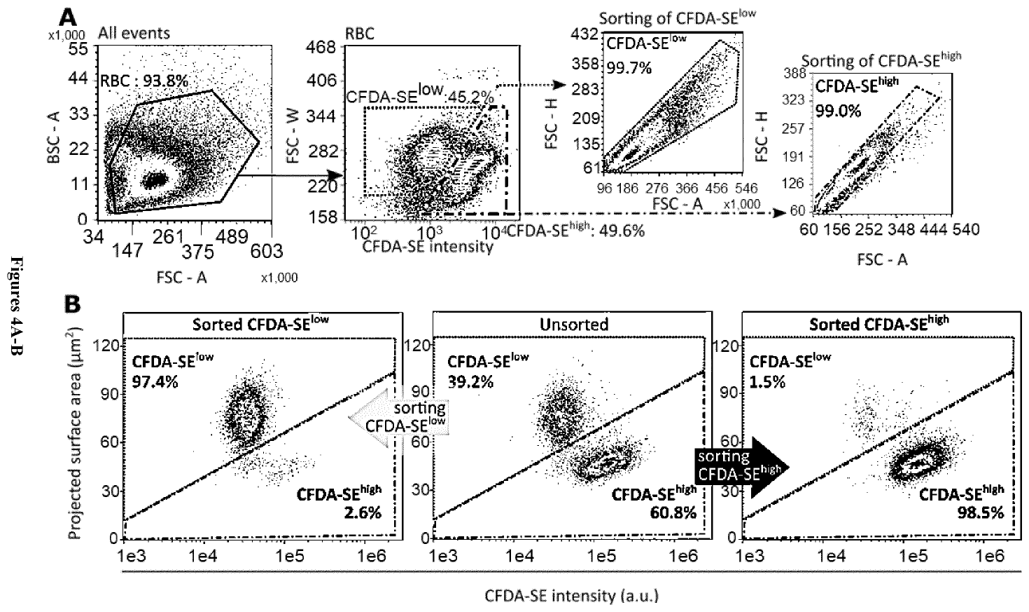

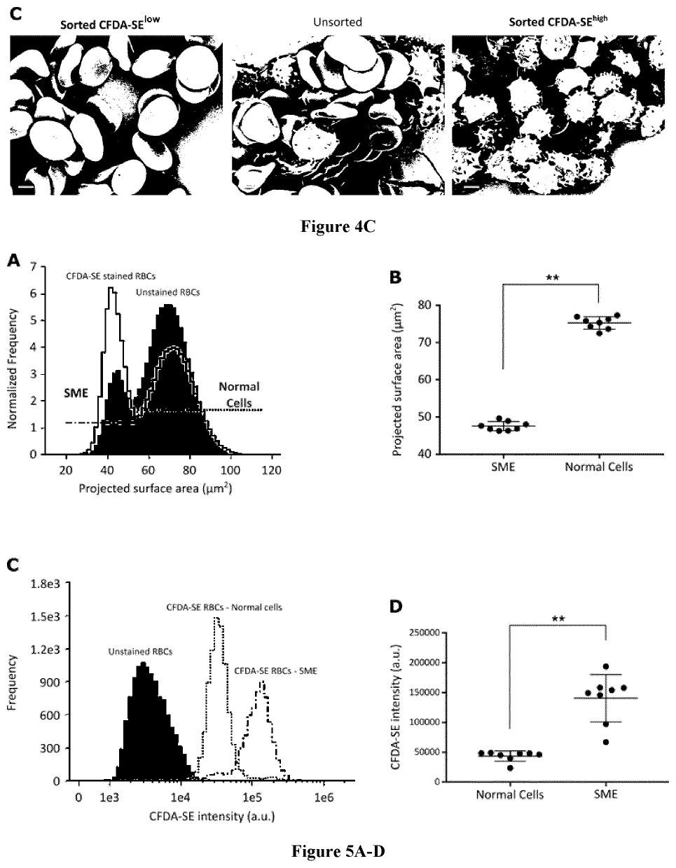

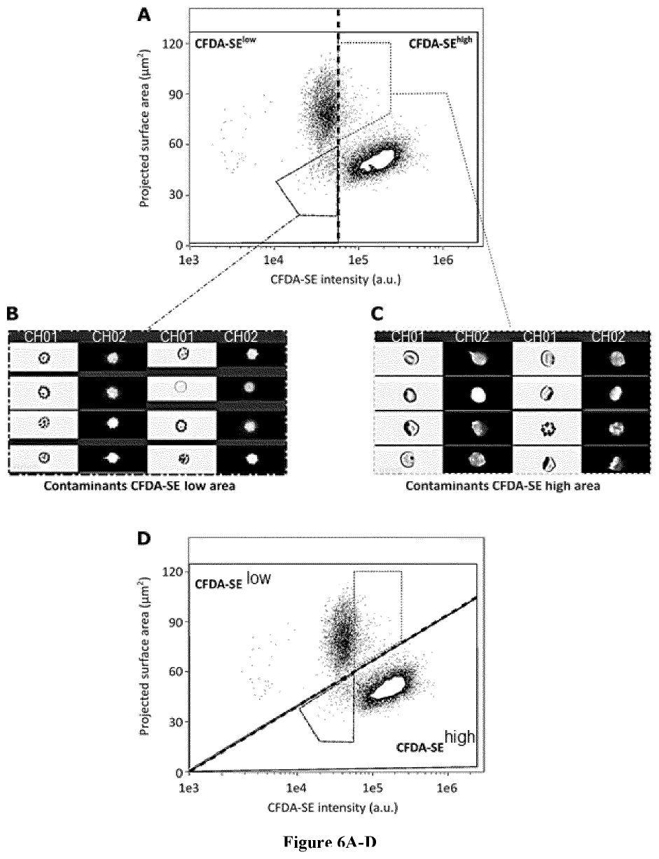

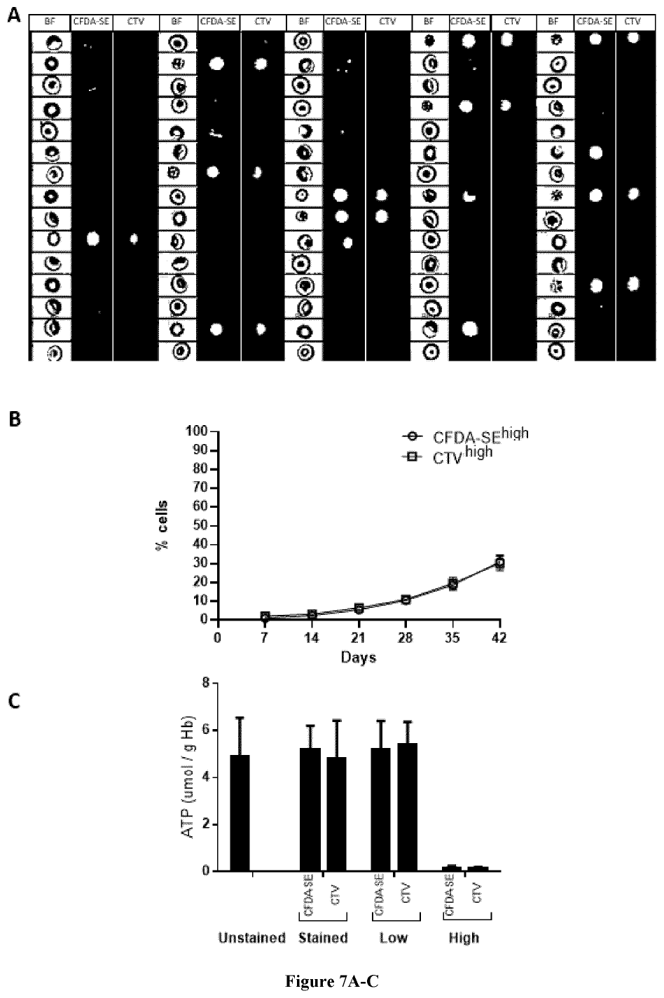

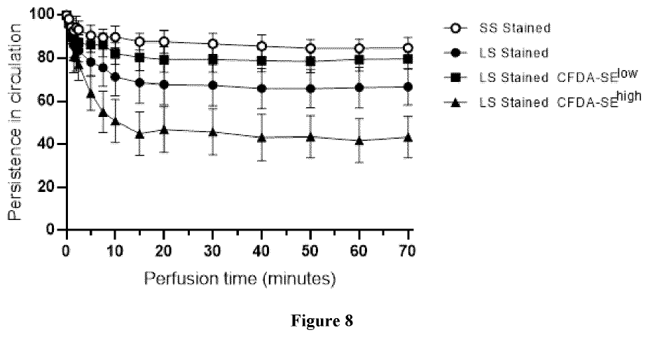

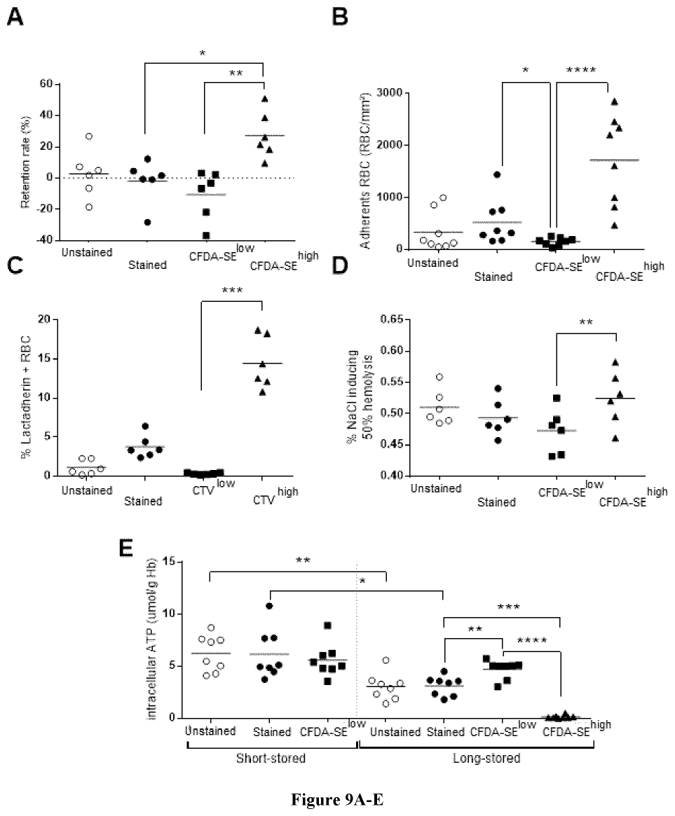

We next used imaging flow cytometry, that enables simultaneous analysis of fluorescence and morphological parameters, on 8 RBC concentrates stored for 42 days. A bimodal distribution of CFSE-DA staining was detected (black line, FIG. 2A) and visible in 8/8 RBC concentrates. Segregating CFDA-SE^{low} RBC (dotted line, FIG. 2A) from CFDA-SE^{high} RBC (dashdotted line, FIG. 2A) was performed independently for each donor, using the nadir of the bimodal frequency histograms as the gating boundary. The mean fluorescence intensity of CFDA-SE^{high} RBCs (154729 a.u.±34931) was statistically higher than for the CFDA-SE^{low} subpopulation (39055 a.u.±6980; p=0.0078; FIG. 2B). We next evaluated the projected surface area of each subpopulation to discriminate morphologically-altered RBC, the storage-induced micro-erythrocytes (SMEs), from morphologically-normal RBC along storage (Roussel, 2017; Roussel, 2021). Comparison of the distribution of projected surface area between the two subpopulations showed that CFDA-SE^{high} RBCs have a lower projected surface area than RBCs from the CFDA-SE^{low} subpopulation (FIG. 2C). Mean projected surface area of CFDA-SE^{high} RBCs (50.1 μm²±2.2) was significantly lower than for CFDA-SE^{low} RBCs (73.7 μm²±1.7; p=0.0078; FIG. 2D). Morphologic analysis of imaging flow cytometry brightfield images showed that the CFDA-SE^{high} subpopulation contained a majority of echinocytes III, spheroechinocytes and spherocytes (FIG. 2E) while the CFDA-SE^{low} subpopulation was composed mostly of discocytes and echinocytes I and II (FIG. 2F). Reciprocally, selection of SMEs and morphologically-normal RBCs (by their projected surface area) confirmed that most SMEs are CFDA-SE^{high} and that morphologically-normal RBCs are CFDA-SE^{low} RBCs (FIG. 5A, B, C, D). The gating strategy to segregate CFDA-SE^{low} and CFDA-SE^{high} subpopulations could be improved by using a size parameter (projected surface area), in addition of CFDA-SE intensity (FIG. 6A, B, C, D).

### CFDA-SE^{high} Staining Enables the Quantification by Flow Cytometry of SMEs Along Storage

We next evaluated the evolution of RBC morphology during storage by flow cytometry using the CFDA-SE staining protocol and compared these observations to the proportion of SMEs detected by imaging flow cytometry on unstained cells (Roussel, 2017; Roussel, 2021). A CFDA-SE^{high} subpopulation (now gated using FSC-W and CFDA-SE intensity) accumulated along storage (FIG. 3A) in the 8 RBC concentrates studied (grey line, FIG. 3C). A SME subpopulation (using the nadir of the projected surface area bimodality) also accumulated along storage (FIG. 3B) in the 8 RBC concentrates studied (black line, FIG. 3C). For all donors, the CFDA-SE^{high} subpopulation accumulated upon storage from 3.3%±1.4% on day 3 to 47.2%±18.8% on day 42 (grey line, FIG. 3C), with marked interdonor variability. Similarly, the SME subpopulation accumulated upon storage from 1.3%±0.8% on day 3 to 38.7%±22.9% on day 42 (black line, FIG. 3C). The proportion of morphologically-altered RBCs followed a similar evolution along storage with both techniques, increasing more rapidly after day 21. The proportion of CFDA-SE^{high} RBCs was slightly higher than the proportion of SMEs at each time point, reaching statistical significance on day 3 and 35 of storage. There was a very strong correlation between the proportion of CFDA-SE^{high} RBCs determined using flow cytometry and the proportion of SMEs determined by imaging flow cytometry (p<0.0001; Spearman r=0.93; r²=0.88; FIG. 3D).

### Enriched Preparations of Morphologically-Normal and SMEs Subpopulations of Stored RBCs can be Obtained by Flow Cytometry Sorting

A flow cytometry gating strategy was next used to sort CFDA-SE^{low} and CFDA-SE^{high} RBC subpopulations (FIG. 4A). Imaging flow cytometry was used to evaluate the content of each preparation, using fluorescence intensity and projected surface area. In FIG. 4B a representative density plots of unsorted (middle panel), sorted CFDA-SE^{low} (left panel) and CFDA-SE^{high} (right panel) subpopulations illustrates the average purity obtained. In 5 separate experiments conducted on RBCs stored 42 days, preparations for CFDA-SE^{low} (left panel) and CFDA-SE^{high} (right panel) contained respectively a mean proportion of RBCs of interest of 96.4%±1.4% and 97.9%±2.0%. Scanning electron microscopy images confirmed that a majority of morphologically-normal RBCs (as discocytes and echinocytes I) were found in CFDA-SE^{low} subpopulations (FIG. 4C, left panel) while a majority of SMEs (echinocytes III, spheroechinocytes and spherocytes) were present in CFDA-SE^{high} subpopulation (right panel) with a mix of these morphologies in the unsorted fraction (middle panel).

### EXAMPLE 2

### Material

### Carboxyfluorescein Diacetate Succinimidyl Ester (CFDA-SE) and CellTrace Violet (CTV) Staining

Staining was performed as described (Marin et al., 2022). RBC were washed once in phosphate buffer saline (PBS) and stained with CFDA-SE (21888, Sigma-Aldrich Chimie) (5.5 million RBC/mL, 0.05 μM CFDA-SE in PBS) for 20 minutes at 37° C. Non-fluorescent CFDA-SE diffuses passively into cells and is rapidly processed by cellular esterases resulting in high fluorescent carboxyfluorescein succinimidyl esters (CFSE) that bind cellular components. RBC were then centrifuged, washed once in RPMIc (RPMI 1640 supplemented with 10% FBS, 1% Antibiotic/Antimycotic solution) to remove excess of CFDA-SE and incubated overnight in RPMIc at 22 million RBC/mL. Following incubation, RBC were centrifuged, resuspended in a fresh RPMIc solution and stored at 4° C. until analysis. CTV staining is performed at 1 μM CTV with exactly the same protocol than CFDA-SE.

### Flow Cytometry Analysis

A flow cytometer (FACScanto II, BD Biosciences) was used to analyze phosphatidylserine (PS), microsphiltration, and spleen perfusion experiments. Analyzes were performed with computer software (FlowJo, V10, BD Biosciences). First, single cells were selected using morphological parameters (FSC-H vs FSC-A). Then CFDA-SE/CTV^{low} and CFDA-SE/CTV^{high} subpopulations were gated according to their size (FSC-W) and their CFDA-SE/CTV intensity (collected in FITC/Pacific blue channel). The proportion (%) of RBCs exposing PS at their surface was determined using a fluorescein isothiocyanate (FITC)-conjugated bovine lactadherin (9-BLAC-FITC, Cryopep). After being washed with phosphate-buffered saline (PBS), 1 million RBCs were incubated with 3.32 μg/mL lactadherin-FITC for 15 minutes at room temperature and analyzed.

### Imaging Flow Cytometry Analysis

IFC was performed by ImageStream X Mark II (Amnis® Flow Cytometry, Luminex, Seattle, WA, USA) to determine RBC dimensions and morphology as described (Roussel et al., 2017). RBC were suspended at 1% hematocrit (Hct) just before acquisition in a Krebs-albumin solution (Krebs-Henseleit buffer, Sigma-Aldrich) modified with 2 g of glucose, 2.1 g of sodium bicarbonate, 0.175 g of calcium chloride dehydrate, and 5 g of lipid-rich bovine serum albumin (Albu-MAX II, Thermo Fisher Scientific) for 1 L of sterile water (pH 7.4). Images (×60 magnification) were recorded (INSPIRE software, AMNIS) by the brightfield and FITC channels to be then processed by a dedicated computer software (IDEAS [version 6.2]; Amnis). Focused cells and single cells were respectively selected using the features gradient RMS_M01_Ch01 and Aspect ratio_M01_Ch01 versus Area_M01_Ch01. Front views were selected using the feature Circularity_Object (M01, Ch01, Tight) and projected surface area was determined using the feature Area_Object (M01, Ch01, Tight). At least 6000 front views of focused single RBC/condition were analyzed. SME proportion and CFDA-SE intensity were determined independently for each donor, using the nadir of the bimodal frequency histograms as the gating boundary.

### Cell Sorting

Sorting of CFDA-SE^{low} and CFDA-SE^{high} cells was performed using MA900 Cell Sorter (Sony) with a 100 μm sorting-chip at the maximum speed of 10,000 events per second in semi-purity mode. Unsorted RBCs were selected using BSC-A vs FSC-A then CFDA-SE^{low} and CFDA-SE^{high} subpopulations were gated according to their size (FSC-W) and their CFDA-SE intensity (collected in FITC channel). Cell doublets were excluded by using FSC-H vs FSC-A and target cells were collected in tubes containing 1 mL of RPMIc, then centrifuged and resuspended in RPMIc to be stored at 4° C. until analysis.

### Microsphiltration

Calibrated metal microspheres 5-25 μm in diameter that form a matrix that mimics flow through interendothelial slits of the spleen were used to assesses the deformability of RBCs. Microsphiltration plates and diluent RBCs were prepared as described previously (Marin et al., 2021) with modifications: diluent RBCs were stained with CellTrace Far Red (C34564, Life technologies) and test RBCs were sorted sub-populations. Mixtures of 5% test RBCs and 95% diluent RBCs are prepared at 1% hematocrit in a Krebs-albumin solution. 200 μL of RBC suspensions (called upstream) were then filtered through the microsphere layer followed by 2 washes of 600 μL Krebs-albumin and downstream suspensions was collected. The upstream and downstream proportion of test RBCs was evaluated by flow cytometry and the retention rate was calculated using the formula:

$$Retention rate = \frac{UP - DW}{UP} \times 100$$

### With:

UP=% of test RBCs in upstream sample
DW=% of test RBCs in downstream sample

Positive values, named retention, indicate that test RBC are less deformable than diluent RBCs, whereas negative values, named enrichment, indicate that test RBCs are more deformable compared to diluent RBC. To validate each experiment, control samples were unstained diluent RBCs (negative control) and 0.8% glutaraldehyde-fixed unstained diluent RBCs (positive control).

### ATP

Adenosine triphosphate intracellular concentration was determined using an ATP assay kit (ATPlite, PerkinElmer). ATP concentrations were normalized against the hemoglobin (Hb) concentration of each sample (μmol/g Hb).

### Osmotic Fragility

RBC osmotic fragility was determined as previously described (Roussel et al., 2017) with modifications to increase sensitivity. Briefly, 0.8 million RBC were washed in PBS, incubated for 45 minutes in hypotonic NaCl-PO4 solutions (equivalent to NaCl solutions, ranging from 0% to 0.9%), and centrifuged (800 g, 5 min). Pseudo-peroxydasic activity of the hemoglobin is revealed by adding 50 μL of 3,3′,5,5″-tetramethylbenzidine (TMB) to 12.5 μL of supernatant for 1 hour. Absorbance was measured at 655 nm using a spectrophotometer and percentage of hemolysis for each salt concentration was calculated (DO value measured for condition Eq NaCl 0% corresponding to 100% hemolysis).

### Dynamic RBC Adhesion on Endothelial Cells

Human microvascular endothelial cell line 1 (HMEC-1) cells were seeded at 10⁸ cells/mL in Vena8 Endothelial+ Biochips (Cellix Ltd, Dublin, Ireland) previously coated with 40 μL of 0.2% gelatin in PBS. Cells were then incubated for 2 h at 37° C., permitting cell attachment and cultured for 48 h using the Kima pump (Cellix Ltd.) RBC were washed in PBS and resuspended at 1% Hct in Hanks buffer supplemented in 0.4% bovine albumin, Ca²⁺, Mg²⁺, and Hepes (1 mM). To initiate adhesion experiments, a first perfusion step was performed (10 minutes, 0.2 dyn/cm2), enabling interactions between RBC and endothelial cells. Then the shear stress was increased each 5 minutes (0.5, 1, 2, and 3 dyn/cm2) to remove less adherent RBC. Brightfield imaging of adherent RBC was performed at ×10 of magnification (AxioObserver Z1, Zeiss). The number of adherent RBC was determined from 10 pictures taken at the end of the 1 dyn/cm2 step for each condition.

### Human Spleen Retrieval and Ex Vivo Perfusion

Spleens were retrieved and processed as described (Buffet et al., 2006). Human spleens were retrieved in the context of the Spleenvivo project approved by the “Ile-de-France II” Institutional Review Board on 4 Sep. 2017 (#CPP 2015 Feb. 5 SM2 DC). All patients underwent distal splenopancreatectomy for pancreatic disease. Spleens were macroscopically and microscopically normal in all cases. Main splenic artery was cannulated, and spleens were flushed with cold Krebs-albumin solution for transport to the laboratory. Spleens were coperfused with long-stored RBCs (stained with CFDA-SE) and short-stored RBCs (stained with Celltrace Far Red according to the same protocol than CFDA-SE) mixed in diluent RBCs (short-stored untreated), at a final hematocrit of 5% to 30% (Krebs-albumin solution), over a 70-minute period at 37° C. Samples were retrieved from the circuit for flow cytometric or imaging flow cytometric analysis. Persistence in circulation was calculated using the following formula: (% stained RBCs in sample/% stained RBCs at T0)×100.

### Results

### CTV^{high} Erythrocytes Correspond to CFDA-SE^{high} Cells that Accumulate During Pretransfusion Storage

CFDA-SE has fluorescent properties that enables its use with a majority of flow cytometers (λ_{ex/em} 492/517 nm). CellTrace Violet (CTV) is a molecule with chemical properties similar to those of CFDA-SE (it covalently binds intracellular proteins) and has different fluorescent properties (λ_{ex/em} 405/550 nm). We thus hypothesized that CTV would also identify SMEs in long-stored RBCs, allowing SMEs detection in a different fluorescence channel. FIG. 7A shows that CFDA-SE and CTV have a similar staining intensity distribution among long-stored RBCs. Most SMEs, identified in the brightfield channel, show high CFDA-SE staining intensity and high CTV intensity while morphologically-normal RBCs have low CFDA-SE and CTV staining intensity. We next evaluated the evolution of RBCs morphology along storage by comparing the proportion of CFDA-SE^{high} and CTV^{high} RBCs by flow cytometry. Quantification of these subpopulations along storage shows that CFDA-SE^{high} and CTV^{high} RBCs accumulate with very similar kinetics (FIG. 7B). We also show that CFDA-SE-stained and CTV-stained subpopulations of long-stored RBCs have similar intracellular ATP level (FIG. 7C). These data confirm that a CTV staining protocol can be used to quantify SMEs during RBCs storage in blood bank conditions.

### Most CFDA-SE^{high} Red Blood Cells are Rapidly Cleared Upon Ex Vivo Perfusion of Human Spleen

When perfused through human spleen, 61% of SMEs were cleared from circulation in 70 minutes (Roussel et al., 2021). We quantified the persistence of CFDA-SE-stained long-stored RBCs (4 RBC concentrates stored for 35-42 days in SAGM solution) mixed with short-stored RBCs (stored for 3-12 days) in circulation during ex vivo perfusion of human spleen (FIG. 8). 33% of long-stored RBCs were cleared from circulation in 70 minutes. Among long-stored RBCs, the mean proportion of CFDA-SE^{high} in circulation decreased by 57% while the CFDA-SE^{low} subpopulation decreased by only 20%. Interestingly, 15% of CellTrace Far Red-stained short-stored RBCs were cleared from circulation in 70 minutes (used as control of the CFDA-SE staining protocol and allowing their quantification by flow cytometry). Persistence in circulation of the long-stored CFDA-SE^{low} subpopulation is thus similar to short-stored RBCs, suggesting that only the CFDA-SE^{high} subpopulation of morphologically-altered RBCs is severely altered during storage and retained in the spleen after transfusion.

### Storage Lesions are Concentrated in the CFDA-SE^{high} Subpopulation of Long-Stored RBCs

We next evaluated selected markers of the storage lesion on highly-enriched CFDA-SE^{high} and CFDA-SE^{low} subpopulations obtained from long-stored RBCs after flow cytometry sorting. Microsphiltration was performed to compare the biomechanical ability of the long-stored CFDA-SE^{high} and CFDA-SE^{low} RBCs to cross the splenic filter (ie, filterability). The proportion (mean±SD) of CFDA-SE^{high} retained in the microsphilters was 38% higher when compared to CFDA-SE^{low} RBCs indicating a reduced filterability of the CFDA-SE^{high} subpopulation (27.54±14.93% vs −10.56±15.78% retention; p=0.007, FIG. 9A). CFDA-SE-stained RBCs showed similar retention to unstained cells confirming that the staining protocol did not impact RBC filterability (−1.92%±13.79% vs 2.68%±15.14%).

Adhesion experiments on endothelial cells showed that density (mean±SD) of adherents CFDA-SE^{high} was increased 11-fold when compared to CFDA-SE^{low} (1720±874 RBC/cm² vs 154±74 RBC/cm², p<0.0001, FIG. 9B). Adherence was similar between unstained and CFDA-SE-stained RBCs.

Proportion of RBCs exposing phosphatidylserine (PS) on their membrane was evaluated by lactadherin staining. Since lactadherin detection by flow cytometry was not compatible with CFDA-SE staining, we used the CTV staining protocol to identify SMEs. FIG. 9C shows that CTV^{high} subpopulation is composed of 14.47±3.33% PS-exposing RBCs, while the CTV^{low} subpopulation contains 0.29±0.10%. The CTV staining protocol had no significant impact on the proportion of PS-exposing RBCs.

Resistance to osmotic stress was evaluated by exposing long-stored RBCs to an osmotic gradient. The CFDA-SE^{high} subpopulation showed increased osmotic fragility when compared to CFDA-SE^{low} RBCs (0.52±0.04% vs 0.47±0.04% respectively, p=0.0014, FIG. 9D). CFDA-SE staining protocol had no impact on osmotic fragility.

Intracellular ATP was measured in short-stored and long-stored RBCs, as shown in FIG. 9E. The long-stored unstained RBCs showed a decrease of intracellular ATP content by 51% when compared to short-stored RBCs (3.03±1.28 μmol/g Hb vs 6.25±1.74 μmol/g Hb respectively, p=0.0021). Among long-stored RBCs, the two CFDA-SE subpopulations had a very distinct ATP content with a mean of 0.12±0.15 μmol/g Hb in CFDA-SE^{high} RBCs and 4.69±0.89 μmol/g Hb in CFDA-SE^{low} RBCs (p<0.0001). CFDA-SE-stained RBCs showed increased intracellular ATP content (3.110±0.936 μmol/g Hb) when compared with CFDA-SE^{high} RBCs subpopulation while it was decreased when compared to CFDA-SE^{low} RBCs, confirming that long-stored RBCs contains two subpopulations of different metabolic activity. Interestingly, long-stored CFDA-SE^{low} subpopulation had similar intracellular ATP content when compared to short-stored CFDA-SE^{low} RBCs (4.693±0.889 μmol/g Hb vs 5.58±1.583 μmol/g Hb respectively), suggesting that only the CFDA-SE^{high} subpopulation is severely altered during storage. The CFDA-SE staining protocol had no significant impact on the intracellular ATP content of short-stored or long-stored RBCs.

The CFDA-SE^{high} subpopulation is preferentially cleared in an ex vivo model of human spleen perfusion. Metabolic and rheological storage lesion markers are concentrated in this morphologically-altered subpopulation, while long-stored morphologically normal cells display properties similar to short-stored RBCs. These data further support that the CFDA-SE staining protocol provides a marker of storage lesion predictive of transfusion recovery.

### REFERENCES

Throughout this application, various references describe the state of the art to which this invention pertains. The disclosures of these references are hereby incorporated by reference into the present disclosure.

1. A method for detecting the presence of storage-induced microerythrocytes (SMEs) in a red blood cell containing composition comprising the steps of i) staining the composition with an amount of a cell permeable dye, ii) incubating the stained composition for a sufficient period of time and under conditions suitable for allowing a bimodality of staining to appear in the red blood cells, and iii) distinguishing the cells by their intensity of staining wherein the subpopulation of SMEs are characterized by a high intensity of staining.

2. The method of claim 1 wherein the red blood cell containing composition is an individual stored red blood cell (RBC) unit.

3. The method of claim 1 wherein the cell permeable dye is a fluorescent dye.

4. The method of claim 1 wherein the cell permeable dye is an amine tracer.

5. The method of claim 1 wherein the cell permeable dye is a succinimidyl ester-based dye that binds to the amine groups of cellular proteins.

6. The method of claim 6 wherein the cell permeable dye is selected from the group consisting of carboxyfluorescein diacetate succinimidyl ester (CFDA-SE), carboxyfluorescein succinimidyl ester (CFSE), carboxyeosin diacetate succinimidyl ester and derivatives thereof.

7. The method of claim 6 wherein the cell permeable dye is carboxyfluorescein diacetate succinimidyl ester (CFDA-SE).

8. The method of claim 1 wherein the cell permeable dye is provided to the cells at a concentration ranging from 0.01 μM to 1 μM.

9. The method of claim 1 wherein the incubation step is carried out at 37° C. and for at least 10, 15, 20, 25 or 30 minutes.

10. The method of claim 3 wherein the fluorescence emitted by the fluorescent dye is measured by means of a flow cytometer.

11. The method of claim 1 that further comprises the step of assessing the size of the cells wherein the SMEs are characterized by a small size and a high intensity of staining.

12. The method of claim 1 that further comprises the step of quantifying the amount of SMEs present in the red blood cell containing composition.

13. A method of assessing the storage quality of a red blood cell containing solution comprising quantifying the amount of SMEs present in the composition by the quantifying method of claim 1 wherein the amount of SMEs correlates with the quality storage.

14. The method of claim 13 wherein the higher is the amount of SMEs present in the red blood cell composition, the worse is the storage quality of the red blood cell composition.

15. A method of predicting the transfusion recovery of a patient transfused with a red blood cell containing composition comprising quantifying the amount of SMEs present in the composition by the quantifying method of claim 1 wherein the amount of SMEs correlates with the recovery yield.

16. The method of claim 15 that comprises the steps of i) quantifying the amount of SMEs present in the red blood cell containing solution, ii) comparing the amount quantified at step i) with a predetermined reference value, iii) and concluding that the patient will a have a good recovery when the amount quantified at step i) is lower than the predetermined reference value or concluding that the patient will have a poor recovery when the amount quantified at step i) is higher than the predetermined reference value.

17. A method of preparing a red blood cell containing composition that is suitable for transfusion comprising i) detecting the presence of SMEs in the red blood cell containing composition by the method of claim 1, and ii) separating the SMEs from the red blood cell containing composition.

18. The method of claim 8, wherein the cell permeable dye is provided to the cells at a concentration of 0.05 μM.

19. The method of claim 9, wherein the incubation step is carried out for 20 minutes.
